(19) United States
(12) Patent Application Publication
(10) Pub. No.: US 2021/0335460 A1
(43) Pub. Date: Oct. 28, 2021

(54) Gateway 2Health (G2H) Remote Monitoring for Managing Type 1 Diabetes ( T1D)

(71) Applicant: Christopher Anthony Noronha, Falls Church, VA (US)

(72) Inventor: Christopher Anthony Noronha, Falls Church, VA (US)

(21) Appl. No.: 17/035,935

(22) Filed: Sep. 29, 2020

Related U.S. Application Data
(60) Provisional application No. 63/016,224, filed on Apr. 27, 2020.

(51) Int. Cl. G16H 10/60; G16H 20/60; G16H 40/67

(52) U.S. Cl. CPC G16H 10/60; A61B 5/4866; G16H 40/67; G16H 20/60

(57) ABSTRACT

A Digital Clinical Management Business Process to manage large numbers or groups of Patients with Type 1 Diabetes (T1D) focused on Glucose levels, Diet, Exercise, Insulin dosage and timing, Carbohydrate and Protein intake. The presentation of 12 clinical parameters including pathology, imaging and prescription details acquired from patients in their homes or through external labs in a single digital format platform defines the process. The Solution is designed for large TID patient numbers with patients connected at their homes through the internet to a dedicated Nurse or Diabetic educator monitoring centre while providing specific features and retaining the ability to highlight clinical variances for individual patients through digital formats. The process enables both the Patient and Health Care Staff to review the effects of changes in critical variables.

### CROSS REFERENCE TO RELATED APPLICATIONS

Provisional Patent Application No. 63/016,224, Dated Apr. 28, 2020, Confirmation Number 1082.

Mr Christopher Anthony Noronha, US Citizen, Falls Church, Va., USA and Western Australia, Australia has developed and invented the Gateway2Health (G2H). Remote Monitoring Solution for Managing Type 1 Diabetes (T1D).

The invention comprises, a specially designed Cell Phone App, linked to a business process which integrates Data on a single platform and displays multiple Digital formats enabling a Clinical transformation consequent to the Digital Management of T1D not achievable by the Human Mind or Computer acting independently.
Please refer to: FIG. 1

Some of the parameters required to monitor Type 1 Diabetes change on a daily basis (Diet, Exercise, Glucose readings, Insulin Dosage) and therefore clinical Digital management on a timely basis is critical. Aside from historical glucose test results, some critical parameters can also be reviewed before the next meal and glucose testing cycle enabling health care staff and the patient to get ahead of the disease and flatten glucose “spikes” or lows (“Hypos”). Please refer to: FIGS. 1, 2, 3

The Solution is designed for large TID patient numbers instead of individual monitoring solutions, with patients connected at their homes through the internet to a dedicated Nurse or Diabetic educator monitoring centre while providing specific features and retaining the ability to highlight digital changes or variances for individual patients through Digital formats. The process enables both Patient and Health Care Staff to review the effects of changes in critical variables.

The actual activities of acquisition and transmission of clinical data and the subsequent integration in a cloud data base together with algorithms highlighting clinical variances are common Technology processes which are used in the invention to generate the process and display data in the desired formats.

The Solution will be used by Doctors, Diabetic Educators and Nurses to manage T1D patients and by patients themselves to develop equity in their own health. Type1 Diabetics comprise around 6% of the total Diabetic population of 30 Million patients in the U.S. but account for an estimated $133 Billion of lifetime medical costs and a disproportionate $289 Billion of other lifetime costs including loss of school days, work days, and income loss aggregating to a total $422 Billion. There is a critical and urgent need to direct resources to the management of this clinical sub segment.

While there are several commercial applications and technologies which enable the Doctor and Patient to transmit their glucose readings and receive clinical advice, this invention and business process is distinct because it is designed to deliver:
An integrated Digital Solution which enables remote management of large T1D Patient numbers in a Community Format while simultaneously highlighting individual patient formats and variances.
A focus on all the 12 variables required to manage Type 1 Diabetes (T1D) set out under Tab [0020] within a single digital platform, variances (changes) from pre set clinical thresholds, diet, carbs, proteins, mean glucose, CV %, pathology, prescriptions and imaging.
Digital Integration of previous clinical readings with other data including a Digital Carbohydrate and Protein Counter and the facility to adjust insulin dosage based on future carb intake which has a direct impact on glucose readings and can be managed prior to the glucose test cycle.
The solution integrates and incorporates the ICR (Insulin Carbohydrate Ratio) and ISF (Insulin Sensitivity Factor) the two critical parameters used by Doctors and Patients together with the Carb Counter to provide Patients and Health Care Staff with the tools to manage diet, insulin dosage and resulting glucose readings.
   Please refer to FIG. 2

To achieve this objective, the inventor Mr Christopher Anthony Noronha has engaged with endocrinologists, physicians, diabetic educators and nurses to design a solution that is not provided in traditional clinical management tools.

The Solution is in operation at 2 Overseas Hospitals directed exclusively to T1D patients and has received positive reviews from Endocrinologists, Physicians, Patients, Diabetic Educators and Nurses. A pilot project of the invention was implemented overseas with results documented internally and shared with the client on May 28, 2020. The results have not been patented or published in a printed publication either in the U.S. or overseas. Ref FIGS. 35,36,37

### STATEMENT REGARDING FEDERALLY SPONSORED RESEARCH AND DEVELOPMENT (If Applicable)

Not Applicable

### REFERENCE TO SEQUENCE LISTING, A TABLE, OR A COMPUTER PROGRAM LISTING COMPACT DISC APPENDIX (If Applicable)

Not Applicable

### Details of Acronyms Used in this Patent Application

a) G2H: Gateway to Health
b) T1D: Type 1 Diabetes.
c) App: Special designed Cell Phone Application
d) CMBP—Clinical Management Business Process
e) IPCDB: Integrated Patient Clinical Data Base
f) MDT: Multidisciplinary Team
g) UPID: Unique Patient Identification
h) LTC Center: Long Term Care Center.
i) HbA1c: A lab test that shows the average level of plasma glucose concentration over the previous 3 months.
j) ICR: Insulin Carbohydrate Ratio: The number of grams of carbohydrate that 1 unit of rapid-acting insulin will cover. This ratio is patient specific and used by health staff.
k) ISF: Insulin Sensitivity Factor: Describes how much one unit of rapid or regular insulin will lower blood glucose. It is used by health staff to determine the amount of insulin to give to correct blood glucose readings that are above target and again, is patient specific.
l) Mean Glucose: Used by Health Care Staff in the management of T1D patients. The average of glucose levels over a pre determined time scale, normally 90 or 180 days.
m) CV %: Used by Health Care Staff in the management of T1D. Coefficient of Variation, arrived at by dividing the Standard deviation of the mean glucose for each patient by the Mean Glucose to remove the volatility.

### BACKGROUND OF THE INVENTION

The Gateway to Health (G2H) will provide the platform to integrate technology and clinical expertise to create a defined Digital Clinical management and business process subsequently contained in comprehensive formats and data displays to remotely manage Type 1 Diabetic Patients. Please Ref FIGS. 1-4 the subject of this patent application.

Overall, Type 1 diabetes patient numbers (T1D) account for approximately 6% of total diabetes patients and affects about 20 million individuals worldwide. Among those younger than 20 years of age, T1D accounts for the majority of T1D cases. The current U.S. prevalence estimate of 1-3 million T1D patients may triple by 2050 due to a rising incidence of T1D.

Worldwide, T1D incidence has been rising by approximately 3% per year, possibly in association with changes in the humoral autoimmune response to islet antigens. Other factors causing the rising incidence include early childhood infections, dietary protein makeup, insulin resistance, and inflammatory factors.

Diabetes costs represent a large burden to both patients and the health care system. However, few studies that examine the economic consequences of diabetes have distinguished between the two major forms, Type 1 and Type 2 diabetes, despite differences in underlying pathologies. Combining the two diseases implies that there is no difference between the costs of Type 1 and Type 2 diabetes to a patient. This assumption is incorrect.

One study in 2010 by the University of Michigan and the Center for Health Research and Policy and CNA (Center for Naval Analysis) examined the costs of Type 1 Diabetes, which are often overlooked due to the larger population of Type 2 patients, and compared. them to the estimated costs of diabetes reported in the literature. Costs of T1D are significantly higher than Type 2 Diabetes. Recent updated figures are furnished at tab [0017]. Updated cost data applicable to T1D however has to be extrapolated from the 2010 figures since there have been few cost specific studies or cost updates since that time.
a) The 2010 study, estimated a T1D population in the U.S. of 1,100,000 (6% of the total diabetic numbers) with lifetime medical costs of $133 Billion and estimated loss of income over their lifetime of $289 Billion. The incidence of T1D commences at an early age, (sometimes from 1½ Years onward) compared to Type 2 Diabetes and the consequential lifetime costs of loss of work days and income are extremely significant
b) Estimates of additional numbers of the T1D population every year from the 2010 study are around 28,430 resulting in $3.2 Billion in Medical Costs and $7.3 Billion in lifetime income loss. These are extremely significant numbers and unfortunately there has not been adequate focus on managing the disease, reducing costs and creating a patient centric approach.
c) Patients with T1D typically suffer from the disease for a longer period of time. Regular maintenance of T1D requires daily insulin shots and constant monitoring, representing a significant lifelong cost and time requirement. For T1D, these long-term effects are likely to spill over to other aspects of their lives with resulting economic impacts particularly in indirect costs. Milton et al. compile and review studies in the literature addressing the social consequences of T1D. They find that children with T1D are more likely to miss school and that the employment outcomes are worse, but school performance and educational attainment remain unaffected.

These are other statistics from the Not For Profit “Beyond Type 1” which update data to 2017 and in some cases reconfirm the above figures for the U.S T1D patient population.
1) Approximately 1.25 million Americans have Type 1 diabetes.
2) By 2050, 5 million people are expected to be diagnosed with Type 1 diabetes in the U.S.
3) An estimated 40,000 people are diagnosed with Type 1 diabetes each year.
4) 200,000 people under the age of 20 years old have Type 1 diabetes.
5) Between 2011 and 2012, 17,900 children and adolescents under the age of 20 were diagnosed with Type 1 diabetes.
6) There was a 21% increase in people diagnosed with Type 1 diabetes between 2001 and 2009 under the age of 20.
7) By 2050, 600,000 people under the age of 20 are expected to have Type 1 diabetes.
8) Among people under the age of 20, non-Hispanic whites have the highest rates of new diagnosis of Type 1 diabetes.
9) There is an additional $14 billion in Type 1 diabetes-associated healthcare expenditures and lost income each year caused through increases of 40,000 patients with T1D each year
10) Less than a third of people with Type 1 diabetes consistently achieve target blood-glucose control levels.
11) Preliminary data from T1 International's 2018 access and supply survey states 1 of every 4 US respondents have rationed insulin due to cost.

Developments in insulin delivery and glucose monitoring technology include Continuous Glucose Monitoring (CGM) and Insulin Pumps which provide the patient with the ability to manage glucose volatility and life style. These features however come at a cost of on average $1500-$1800 representing Co Pay for the Devices and $1200-$1500 per year co pay on consumable supplies including tubing for the Pumps and $370 for the CGM. The Cost comparison however is just one factor, with the critical issue being the ability to simultaneously monitor groups of patients to lower unit costs. Please refer Prior art at FIGS. 14, 15 and 16.

Given the cost of insulin, medical supplies and the necessity to provide for emergencies, any solution has to provide a low cost, low technology digital monitoring program designed for simultaneous monitoring of large numbers of patients that is patient centric, and enables the patient to develop equity in the management of their own health through a device that is readily available—their Cell Phone.

The 12 Parameters to successfully manage T1D are as follows:
1) The time and results of the glucose test from patient glucometers (on average 3-4 times a day)
2) The Brand of Insulin—T1D Patients inject both fast acting and normal insulin on the same day.
3) The time of injecting the specific insulin brand and the dosage.
4) Brand of Tablets if prescribed in addition to the Insulin & the time they are taken
5) Exercise schedule details through the Cell Phone Application linked through the UPID
6) Diet using commercially available Carb and protein counters.
7) Insulin Carbohydrate Ratio (ICR)—the dosage of insulin required to manage glucose levels for a specific Carb intake.
8) Insulin Sensitivity Factor (ISF)—dosage of insulin required to keep glucose levels within the target range after considering the ICR.
9) GIR (Glucose in Range): Calculation of the cumulative percentage of glucose test readings that are within the specified range of 70 to 180 mg/dl or a tighter range of 70-154 mg/dl. A higher GIR percentage implies the greater probability of an Hba1C result within target levels. The Hba1c test determines the average level of blood sugar over the past 90 days with the target Al c level for people with diabetes usually 7%. Additionally Mean glucose and the CV % are metrics used by Health Care staff in management of T1D.
10) Prescriptions. (Basic Data and Updates) Direct to the Cloud Data Base either manually or Electronically linked through the Patient ID.
11) Pathology data (Bi Annual) Direct to the Cloud Data Base either manually or Electronically linked through the Patient ID.
12) Retina Scans (Annual): Direct to the Cloud Data Base either manually or Electronically linked through the Patient ID.

The low cost business process enables the T1D patient to develop equity in the management of their own health in their homes, improves quality of life while simultaneously reducing use of high cost Hospital or ER resources.

It is especially directed to clinics, health centers or hospitals in rural or remote regions in the U.S especially economically disadvantaged population segments in addition to developing or emerging overseas markets. In both these geographies, the primary objective is to simultaneously and concurrently, effectively and cost efficiently manage large and growing T1D populations which represent a significant cost burden to these economic regions.

Mr Christopher Anthony Noronha has already developed the necessary Cell Phone and Clinical Data application interfaces linked to the Cloud Data Base and comprehensive Digital formats and they are working at two Overseas Hospitals managing T1D patients aged from 7 Years through 34 Years using a Multidisciplinary team (MDT) of Endocrinologists, Diabetic Educators and Nurses. Please refer to FIG. 3

The MDT are provided with the clinical management tools including access to the Integrated Patient Clinical Data Base (IPCDB) and the ability to remotely manage variances from clinical standards and thresholds. These features are the solution to delivering and managing large numbers of patients using an integrated model of care for T1D. Delivery is at the low cost Home, reducing high cost hospital admissions, re-admissions and use of the ER.

The key factor remains timely and easily available information at a single digital access point highlighting changes of the parameters to enable health care staff to achieve the clinical transformation in patients not achievable by the Human Mind or Computer operating independently.

The invention addresses each of the above points, meets the strategic initiatives of reducing costs in delivery of health care at low cost locations and ensures that the patient creates equity in the management of their own health.

References:

Beyond Type 1 statistics: CENTERS FOR DISEASE CONTROL AND PREVENTION. NATIONAL DIABETES STATISTICS REPORT, 2017. ATLANTA, GA.: CENTERS FOR DISEASE CONTROL AND PREVENTION, U.S. DEPT OF HEALTH AND HUMAN SERVICES; 2017.

### PMCID: PMC2901386

### PMID: 20634976

Published online 2010 Jul. 9. doi: 10.1371/journal.pone.0011501 Estimating the Cost of Type 1 Diabetes in the U.S.: A Propensity Score Matching Method.

### Center for Health Research and Policy 2010.

Betty Tao,¹, *Massimo Pietropaolo, ²Mark Atkinson, ³Desmond Schatz, ⁴and David Taylor⁵

### BRIEF SUMMARY OF THE INVENTION

Approximately 1.25 million Americans (as at 2010) have Type 1 diabetes, By 2050, 5 million people are expected to be diagnosed with Type 1 diabetes. requiring immediate and urgent action to address this situation.

Please refer to FIGS. 1-4

The Clinical Management Business process (CMBP) forms the basis of an integrated model of care for T1D that consists of delivering patient clinical data comprising, Data, Text and Imaging to an Integrated Patient Clinical Data Base (IPCDB) The transmission and integration of data in the IPCDB and clinical algorithms to highlight variances from preset thresholds and identifying patients who “Did not Test” are standard technology processes used to display the desired formats. The presentation of the multiple variables (12 Parameters) in a management format hosted on the G2H website with clinical data acquired from patients in their homes or external labs permits clinical management. Some of the parameters change on a daily basis ((diet, exercise, glucose readings) and therefore clinical management on a timely basis is critical. All data is transmitted after encryption and later Securely stored in the cloud through a secure and encrypted UPID (Unique patient ID).

### Ref FIG. 4

STEP 1: The Remote Health Application is downloaded to the patients Cell Phone. Glucose readings using the patients Cell Phone.
STEP 1: Glucose readings, Insulin Brands, Dosage, Time, are manually entered by patients to an application on their Cell phone & transmitted through the UPID to the data base in the cloud. Diet and Exercise details are included after reference to a Carb and protein counter, the Insulin Carb Ratio and Insulin Sensitivity Factor.
STEP 2: Images—Retina Scans, Prescriptions & Pathology are uploaded to the cloud linked to the Patients UPID. Manually or Electronically.
STEP 3: Data Base in the cloud
STEP 4: Physician, Nurses/Diabetes Educator can access the Patient Data from the cloud and act on variances from normal.
The format comprises Glucose readings after reference to the ICR and ISF, Insulin brand, dosage, Insulin injection Timing, Pathology, Diet, Exercise, Prescriptions and Imaging.
Other formats include a “Daily Dashboard” of all individual patient variances from pre set thresholds and a listing of patients who “did not test” Please Refer to FIGS. 12 and 13

Clinical Management Business Process. Please refer to FIG. 5 Flatten the spikes and Hypos (Low Readings), Focus on Diet, Exercise, ICR, ISF and Refer to GIR %.

Stay ahead of the disease.
STAGE 1 Review of Daily Dashboard variances by Diabetic Educators, Physicians and Nurses
STAGE 2 Call & Message Patients re Insulin Dosage, Carb Ratio, ISF, Diet, Exercise. Advice to deal with Hypos (low) or Hyper (High) or “Did not Test”. Use ICR & ISF with the Carb Counter to manage Diet and get ahead of Insulin Spikes or Hypos (Low Reading).
STAGE 3: Review persistent variances, suggest revised insulin dosage, diet suggestions.
Monitoring Center flattens the spikes and hypos through changes in Insulin dosage if needed using ICR and ISF. Diet & Exercise review.
STAGE 4: Management of patients with abnormal readings, GIR below 60%. Phone or Video Visit.meet Educators and Physicians. Monitoring Center flattens the spikes and hypos through Phone consults on those patient segments with GIR<60%. Focus on Diet, Exercise, Insulin dose.
STAGE 5: Continuous monitoring Stages 1-4 continues. Schedule Pathology & Scans

Physicians, Diabetic Educators and Nurses managing T1D patients need precise information several times daily with respect to The Glucose test time, Insulin Brand, Insulin dosage, Time of dosage, Insulin metrics including the Insulin Carb Ratio and Insulin Sensitivity Factor all contained within one component of the clinical data flow from the Patient cell phone They also need daily information on Exercise and Diet linked to a Carb and protein Counter to adjust insulin dosage based on the patients diet.

Please refer to FIG. 6

The Screen Shot is a working example of Previous records of Diet, Exercise, Insulin Dosage and Glucose levels. In this case since the Carb intake is 48 against a target of 60 and pre meal insulin of 115 mg/dl is below the target of 120 there is no necessity to adjust insulin dosage corresponding to Carb Intake
The Glucose test results at 9 AM-2 hours after having breakfast and an Insulin shot of 11 Units at 6.30 am, exercise of 30 Minutes at the Gym and breakfast at 7 AM show a glucose level of 128 mg/dl.
A screen shot of the screen on the patients cell phone shows some of the data which is generated, an example of the critical insulin calculations based on the Carbohydrates at each meal. FIG. 8 which is viewed by Doctors, Educators, Nurses and Patients themselves. We make the point that the process is designed to be patient centric and patients normally complete all steps on the Cell Phone App within 60 seconds after a days practice

Please refer to FIG. 8 representing One View of the Secure Comprehensive Clinical Format viewed by Doctors, Educators, Nurses and the Patient—Hosted on the G2H Website. With encrypted data from the Cloud Data base displayed in the format.

Clicking on the other Tabs above gives Health Staff access to Imaging, Prescriptions, Pathology in addition to the clinical parameters through the cell phone which change on a daily basis (diet, exercise, glucose readings, insulin dosage) and therefore enables clinical management on a timely basis. The integration of all the 12 parameters and calculation of GIR %—(Glucose in Range) generated several times a day, “Daily Dashboard” and “Did not Test” Refer FIGS. 12, 13 also incorporating the ICR and ISF with the Carb Counter and variable Insulin Dosage is a major product differentiation and is the solution to reducing Hospitalization and Readmissions by managing treatment at the low cost home for T1D patients.

Importantly the solution extends health care services to relatively underserved T1D populations addressing health disparities. The strategy is to deliver Health Solutions linked to data driven outcomes and highlight variances, creating in the process a Clinical Digital Management System. Common standard technology solutions are used to transmit, integrate data and highlight variances as part of the CMBP format and provide the platform to escalate levels of care if variances persist.

Clinical data from multiple patients, Physician practices or Hospitals transferred to the Cloud Data Base can then be viewed in formats to form the basis of community health management with the flexibility of focusing on individuals if required through Location or Project ID (Community Health) or the UPID (Individual Patients)
Please refer to the comparison of features of the invention compared to Prior Art at FIGS. 14, 15 and 16.

### BRIEF DESCRIPTION OF SEVERAL VIEWS OF THE DRAWING

The Clinical Management Business process (CMBP) forms the basis of an integrated model of care for T1D that consists of delivering patient clinical data comprising, Data, Text and Imaging to an Integrated Patient Clinical Data Base (IPCDB). The transmission and integration of data in the IPCDB and clinical algorithms to highlight variances from preset thresholds and patients who “Did not Test” are standard technology processes used to display the desired formats. The presentation of the multiple variables (12 Parameters) in a management format hosted on the G2H website with clinical data acquired from patients in their homes or external labs permits clinical management and is the subject of this invention. Some of the parameters change on a daily basis (diet, exercise, glucose readings) and therefore clinical management on a timely basis is critical. All data is transmitted after encryption and later Securely stored in the cloud through a secure and encrypted UPID (Unique patient ID).

The 12 Parameters are: 1) Time of Glucose Test, and Glucose level 2) Insulin Brand 3) Insulin Dosage & Time 4) Brand of Tablets 5) Exercise details 6) Carbs and Proteins 7) Glucose in Range (GIR %) 8) ICR, 9) ISF 10) Pathology 11) Prescriptions 12) Annual Eye Scans (Imaging)

FIGS. 1, 2, 3, 4, 5

The G2H for T1D invention will provide remote health care services to T1D patients at their homes and as they become older at either their homes or Long Term Care Centers (LTC). The solution is based on use of the Internet, or Cell phone connectivity and monitoring can be therefore be carried out by health staff at any location.

FIGS. 6 and 7.

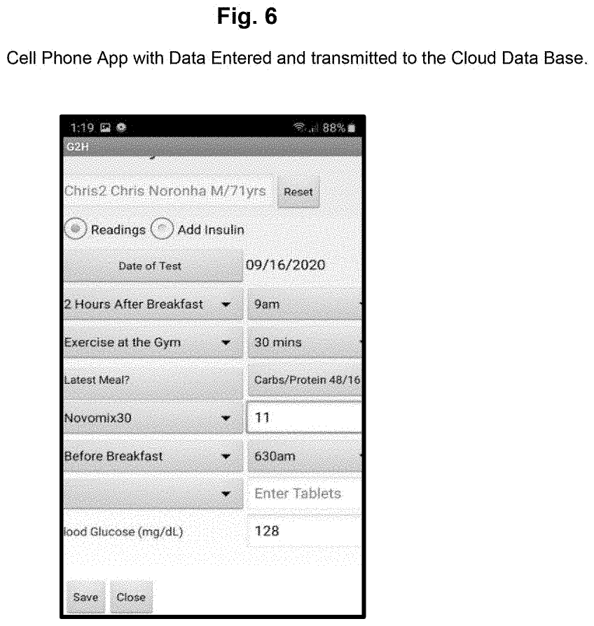

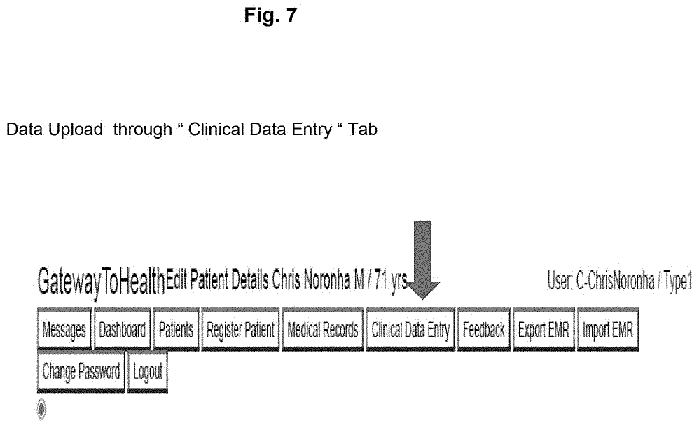

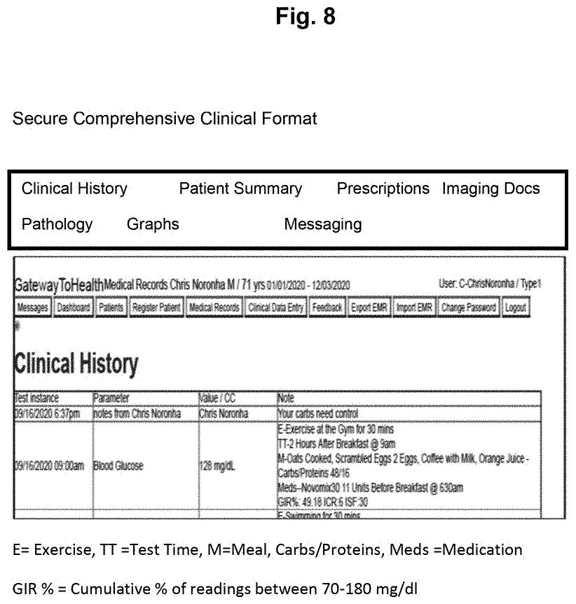

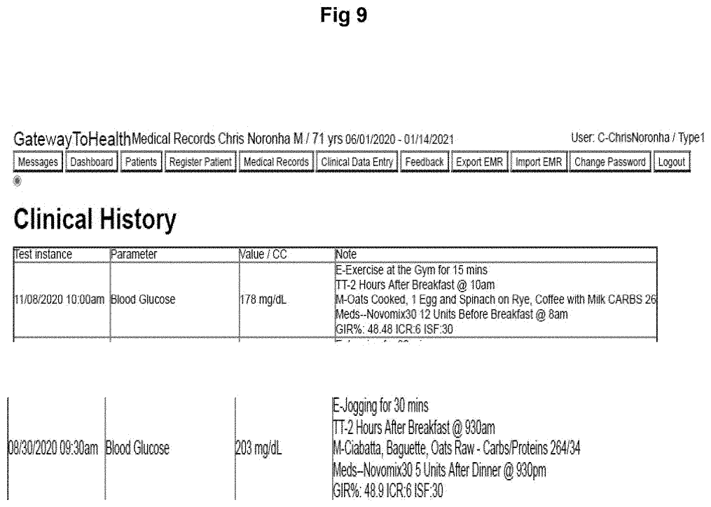

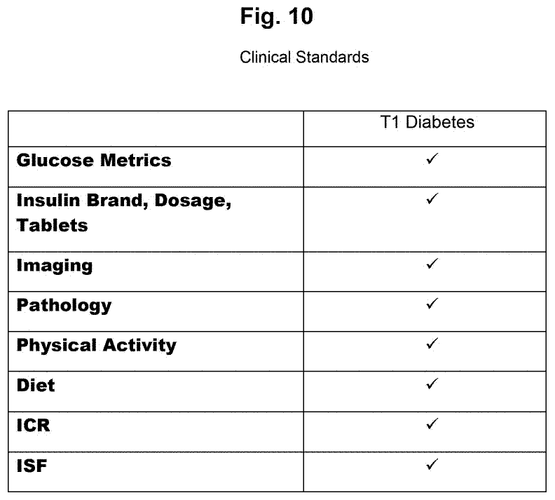

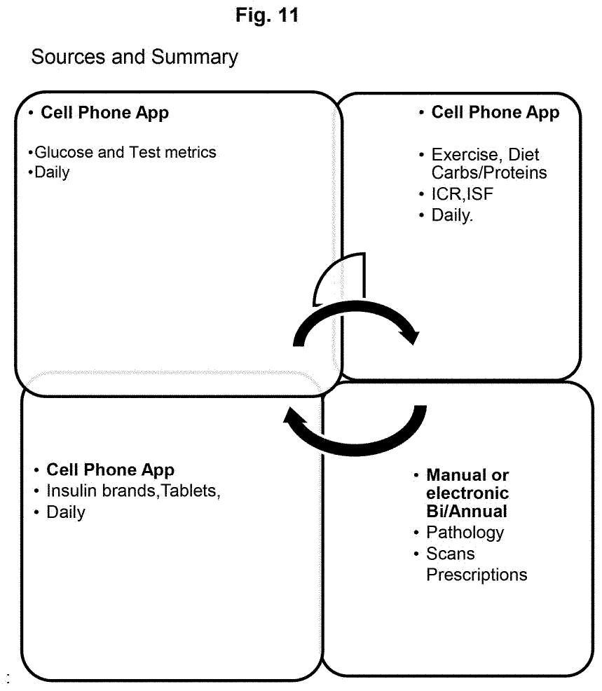

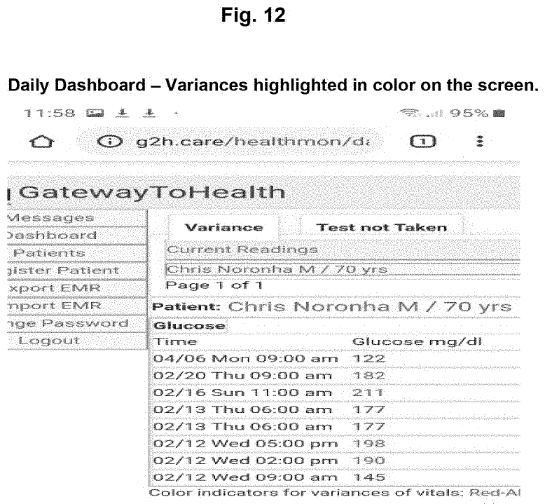

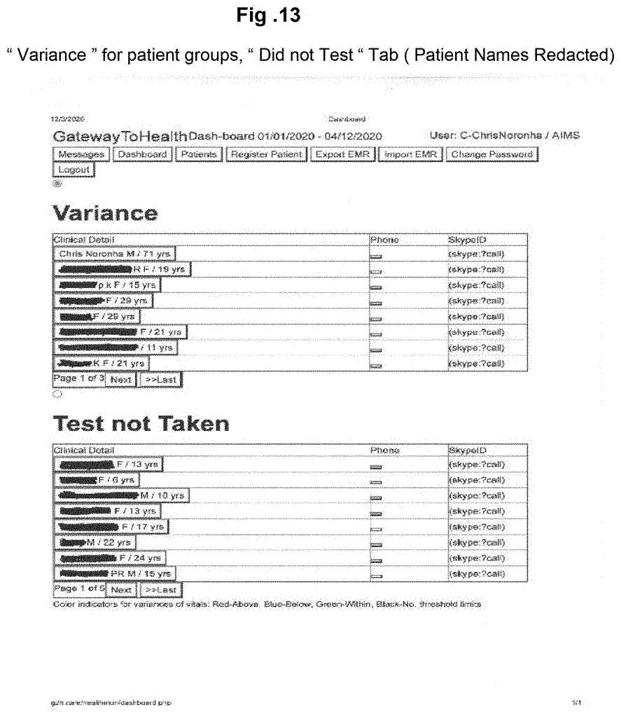

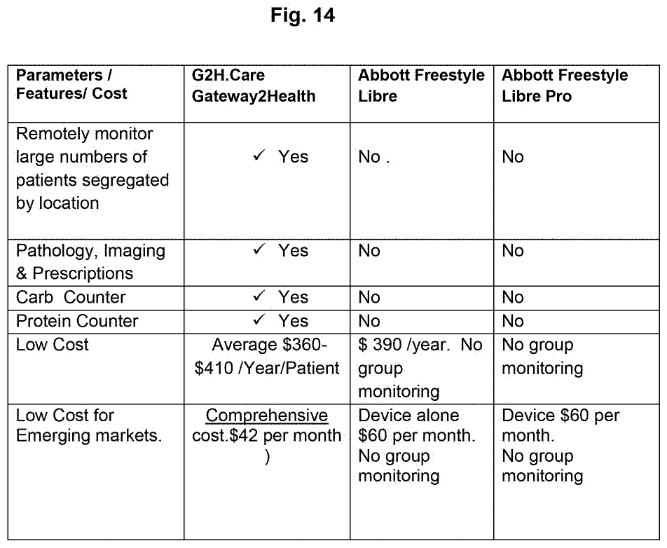

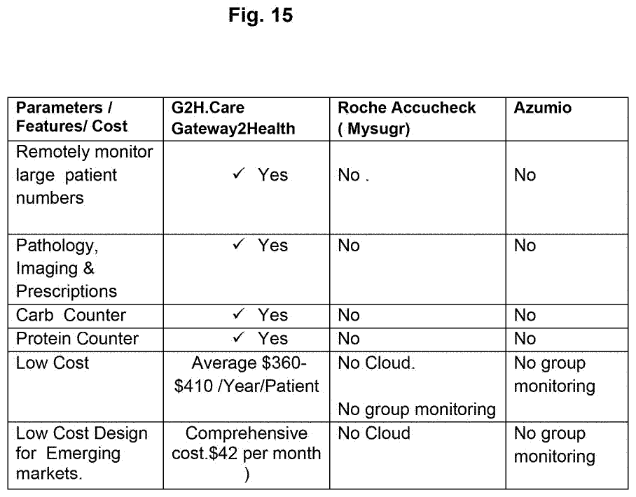

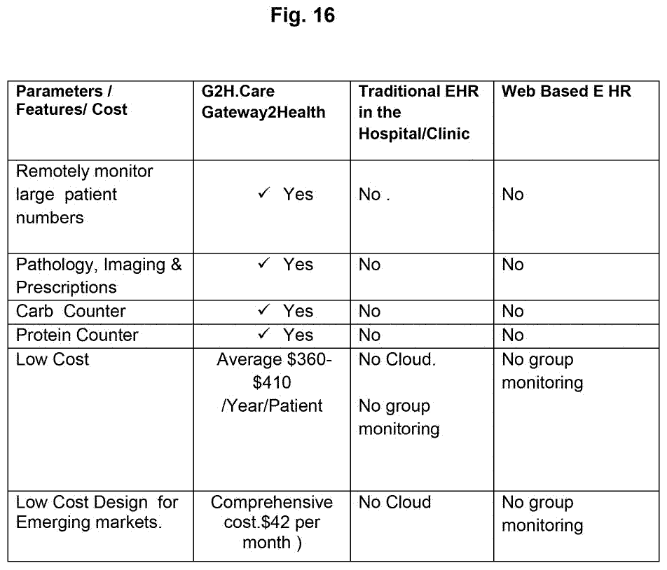

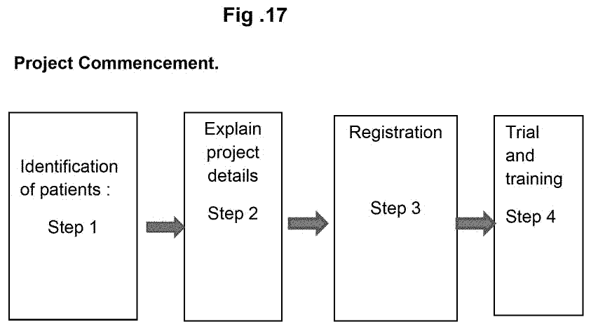

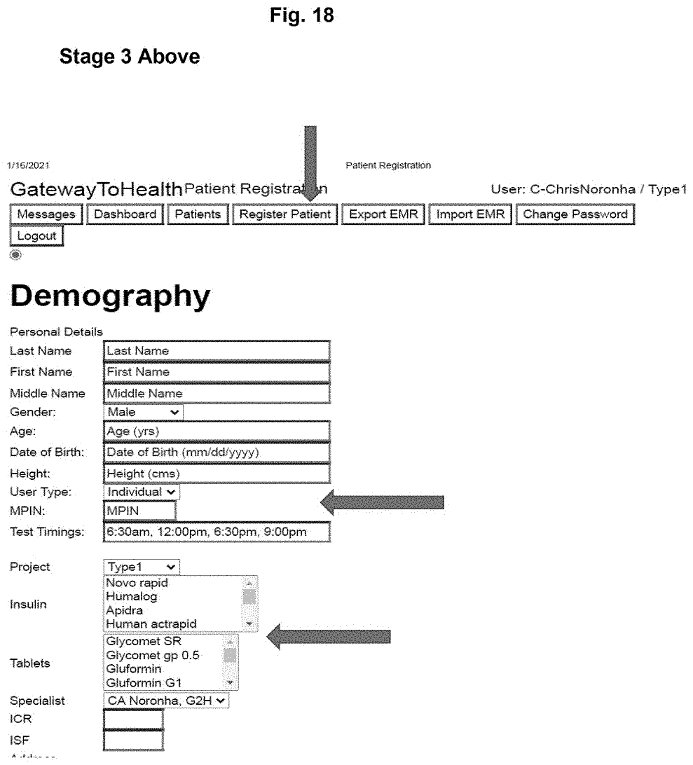

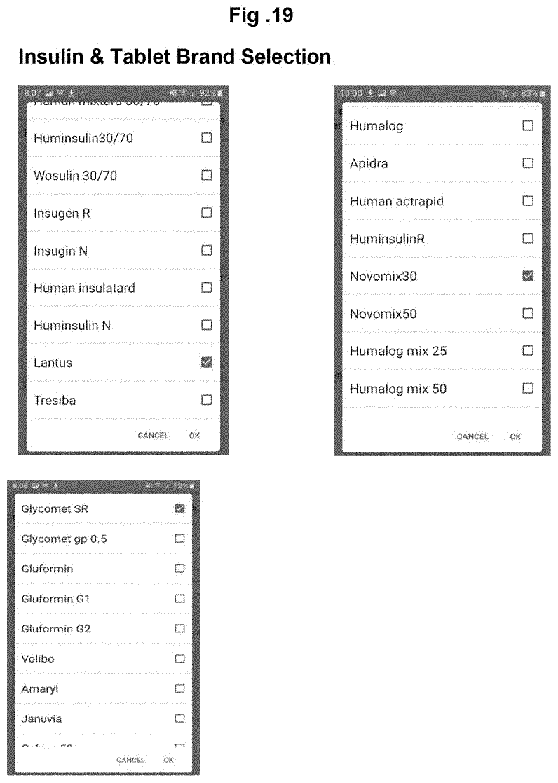

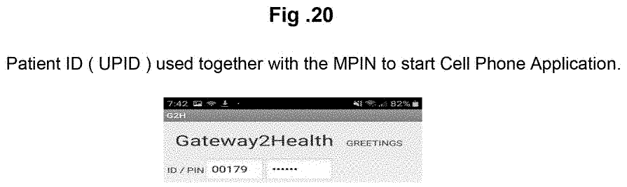

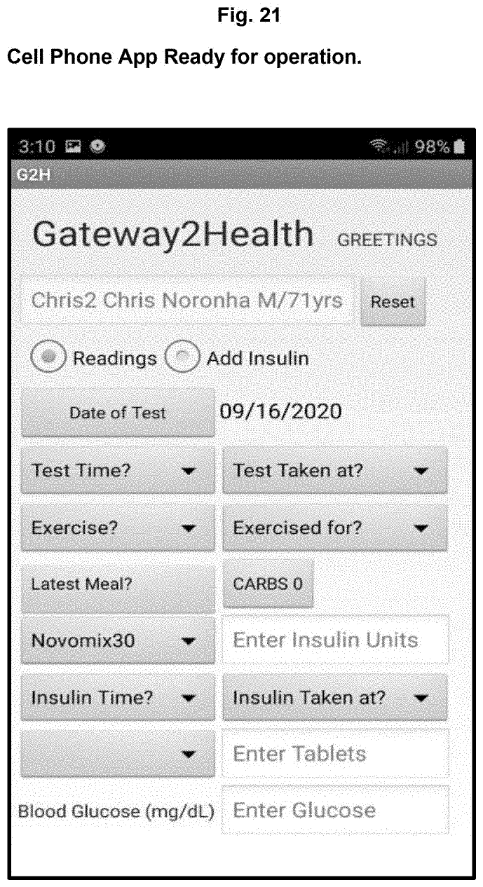

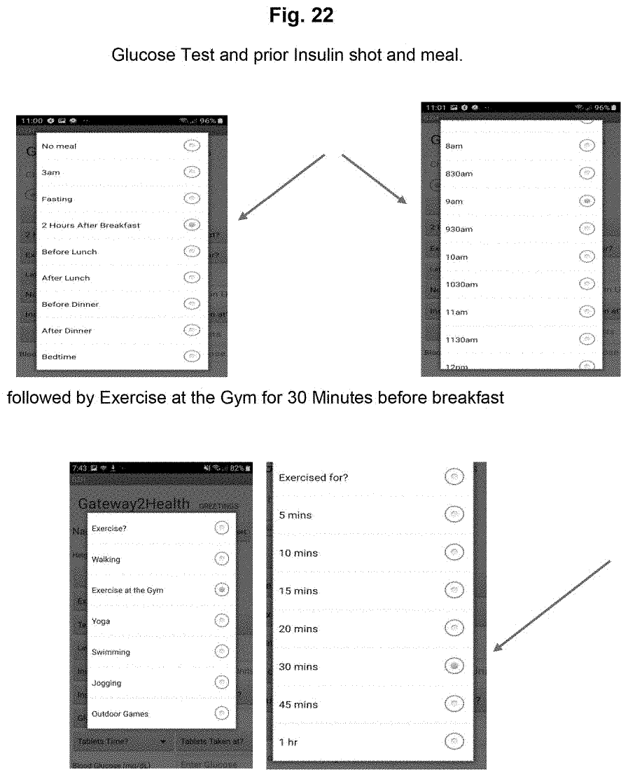

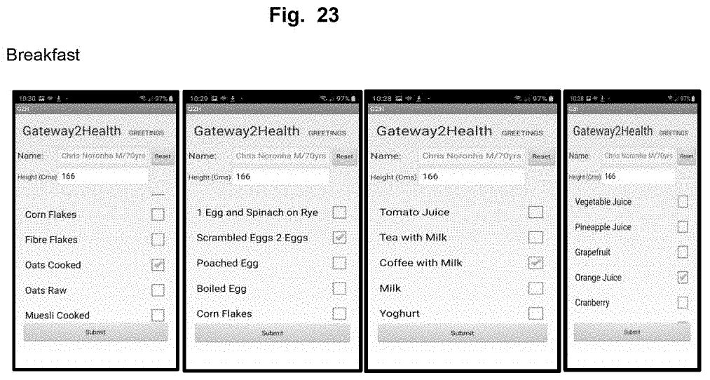

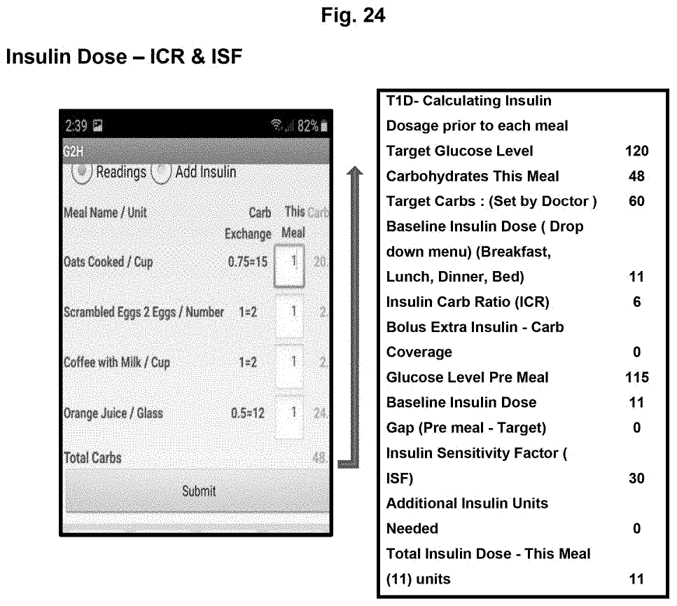

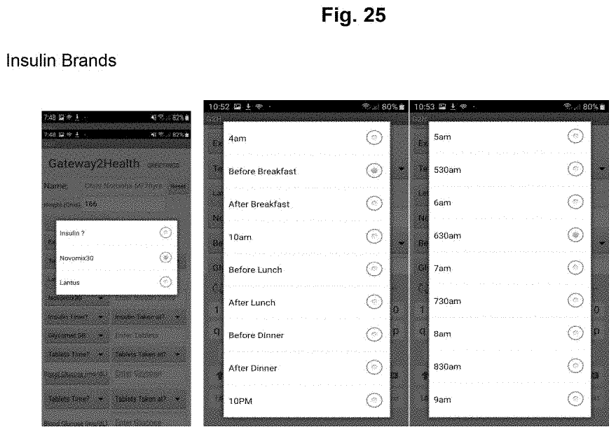

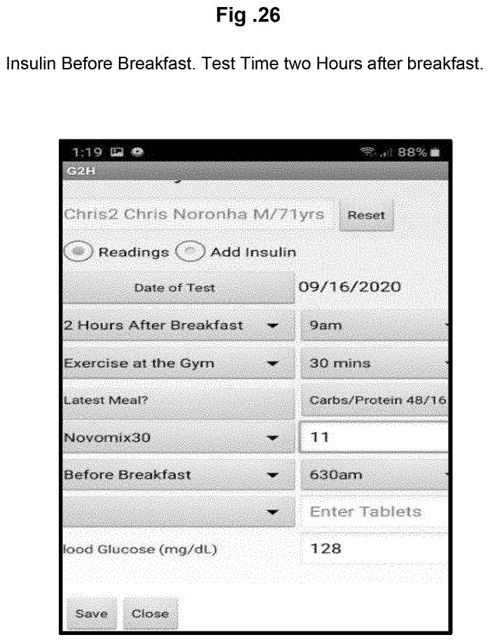

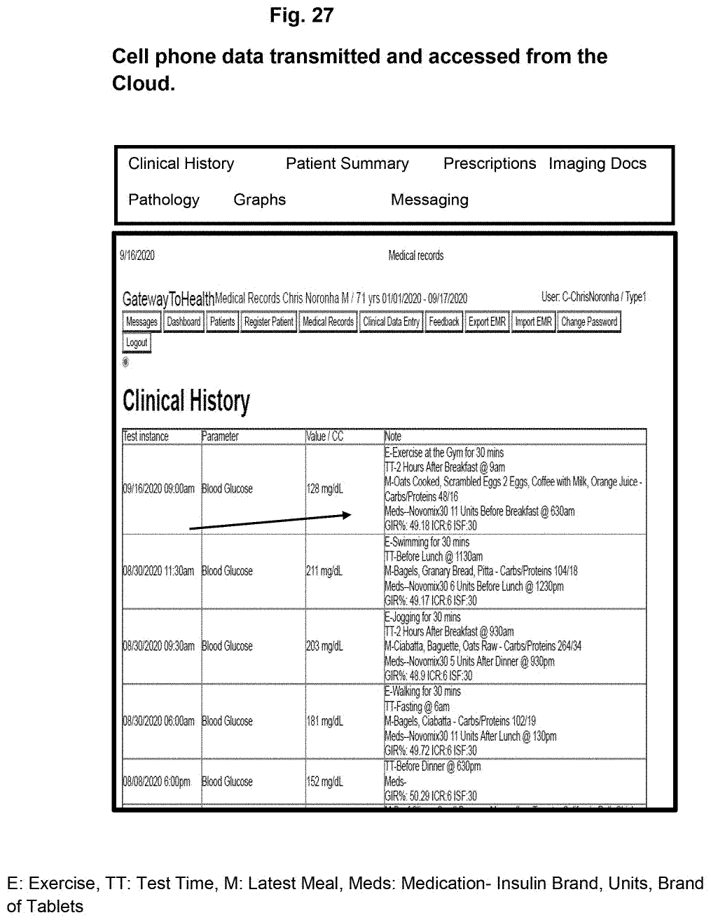

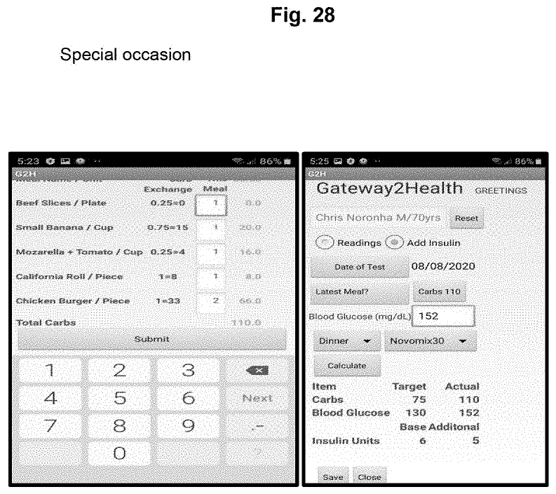

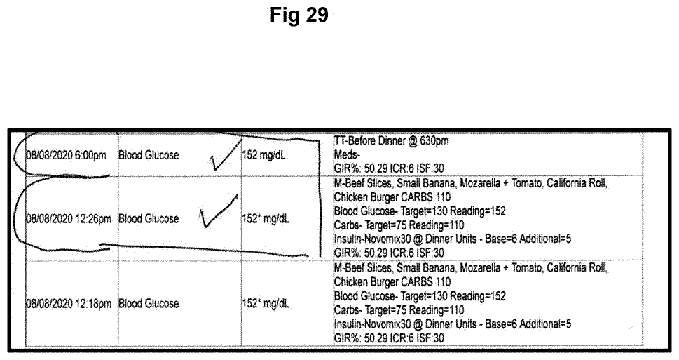

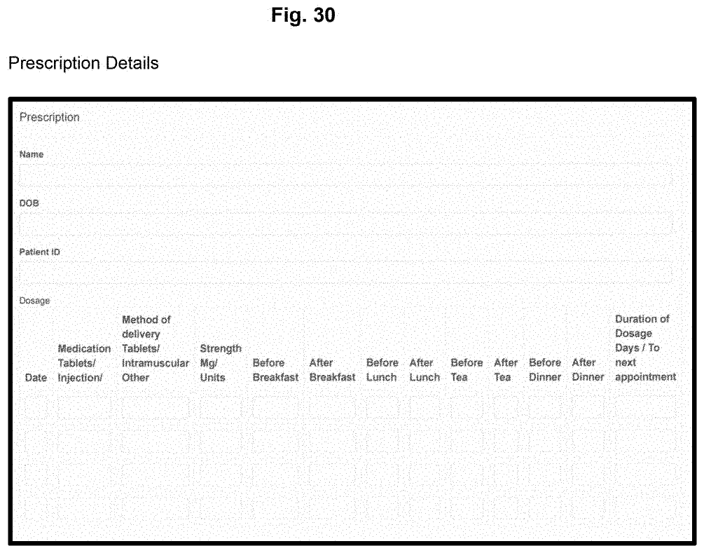

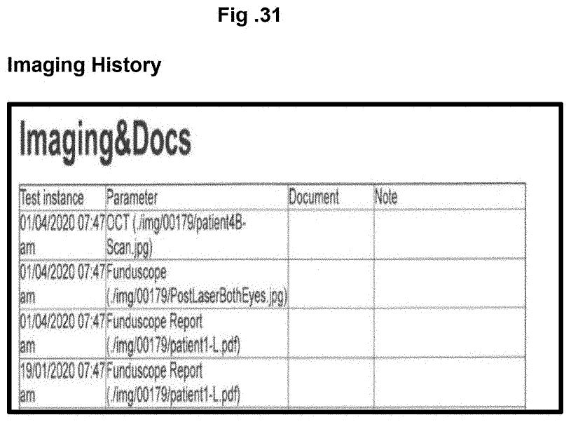

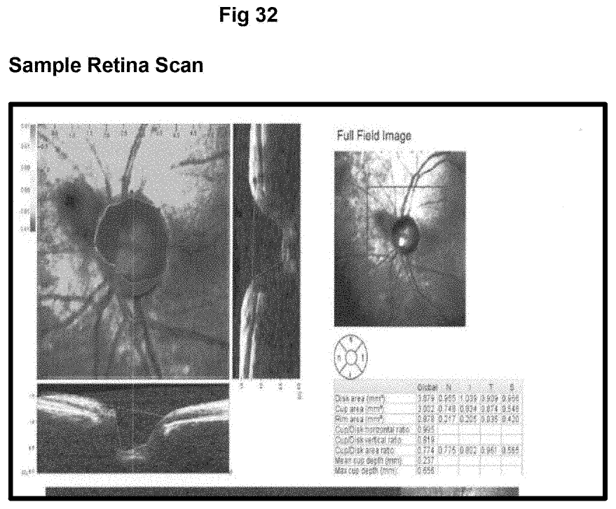

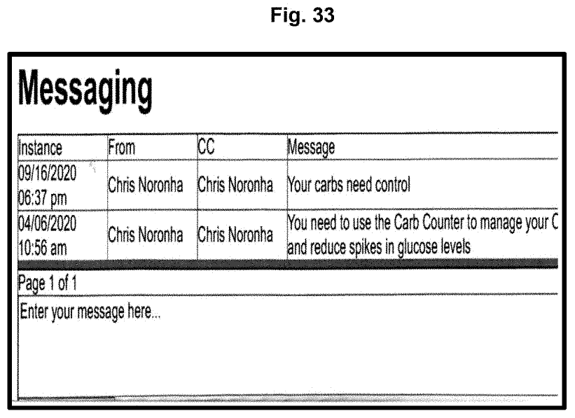

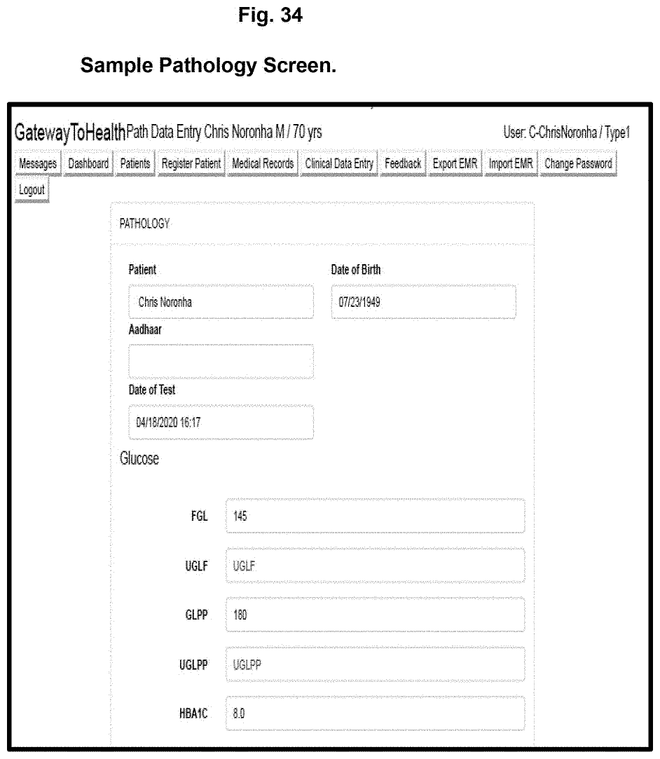

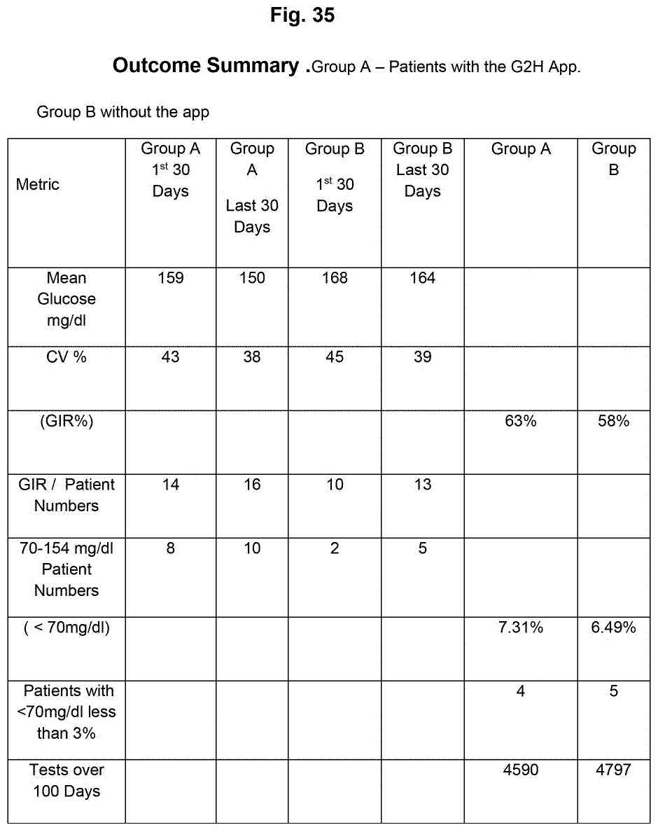

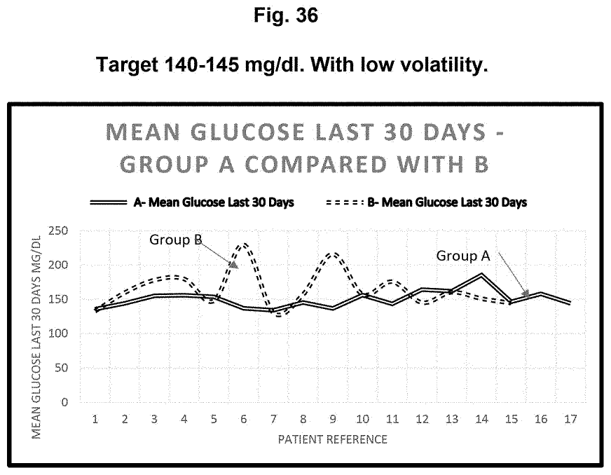

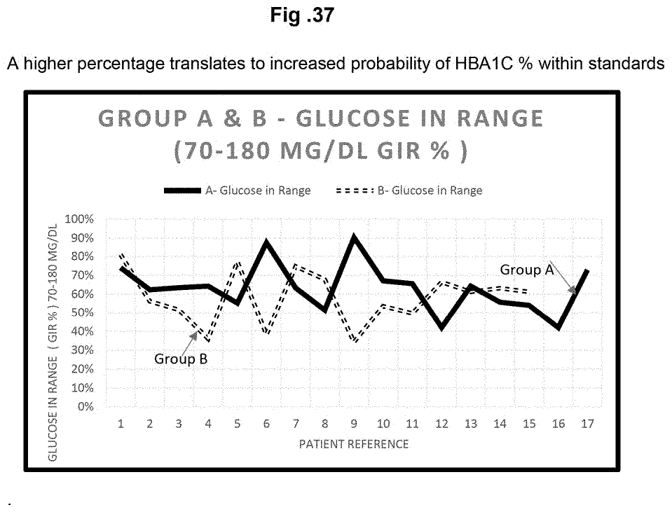

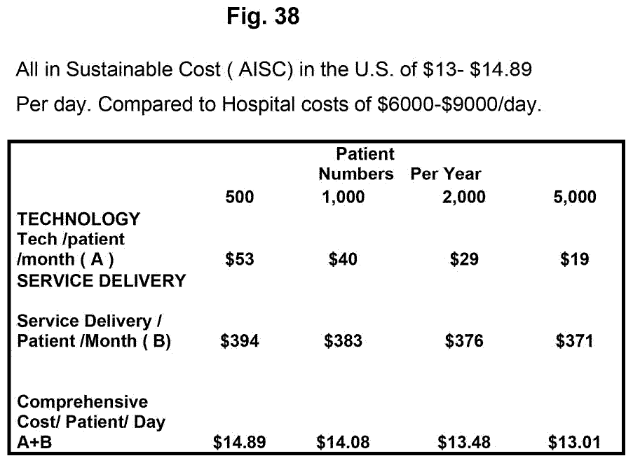

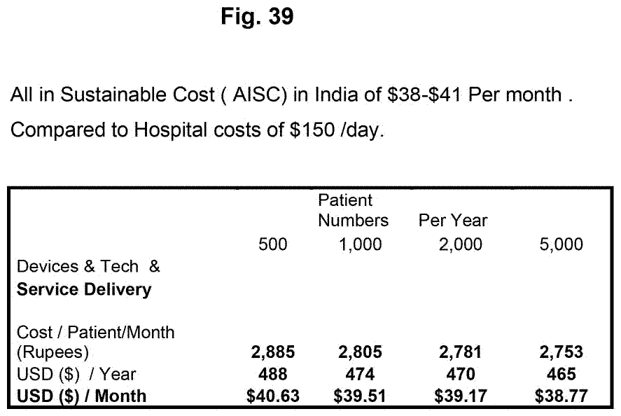

Transmission of daily clinical data is made through a Cell Phone equipped with a proprietary application (FIG. 6) which acquires the data and transfers it to the IPCDB identifying the patient through a Unique Patient ID. The second source of data including Pathology Results, Prescriptions and Images from Retina scan, are transmitted manually or through electronic upload and saved to the Data Base after registration and identifying the patient through the (UPID) through the ““Clinical Data Entry” Tab. Ref FIG. 7 later displayed using the Comprehensive Format for Doctors and Patients on the G2H Web Site.

FIGS. 8 and 9: This Comprehensive Digital format on the G2H website is the principle mechanism for Doctors, Health Care Staff and the Patients themselves to access and manage patient data. It includes tabs for Registration, Daily Dashboard, Exporting or Importing data, Medical Records (including Glucose levels, Diet, Exercise, Insulin and ICR, ISF, Calculated GIR, Pathology, Images) and Messaging. Doctors and Health Staff in a project have access to all patient data while patients have a UPID and MPIN to review their own data. Data Security is built in at all stages of the process at the Cell Phone and the cloud, through encryption and encrypted data security protocols, the UPID and a user selected MPIN. Ref FIGS. 8 and 9.

FIG. 10

The G2H solution will provide Endocrinologists, Physicians, and the MDT with access to the Comprehensive Digital Format hosted on the G2H website using data accessed from the IPCDB (Integrated Patient Clinical Data Base) comprising the 12 variables required to manage T1D. Clinical standards at FIG. 10 set out the necessity of an integrated Patient Clinical Data Base to manage T1D.

FIG. 11

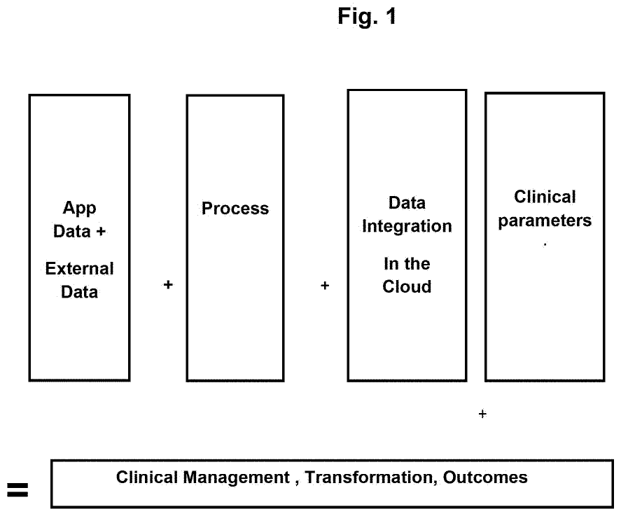

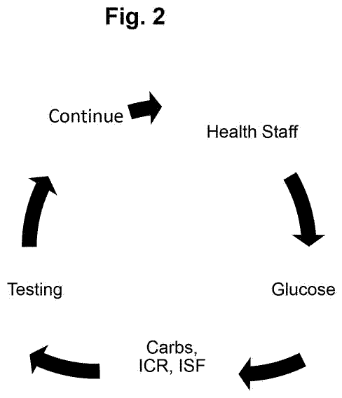

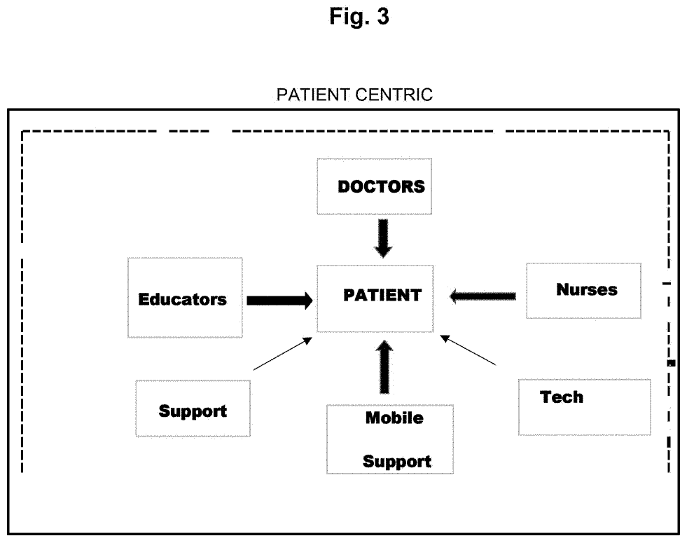

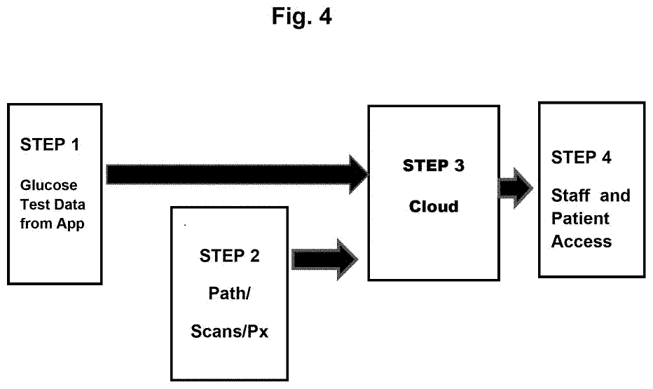

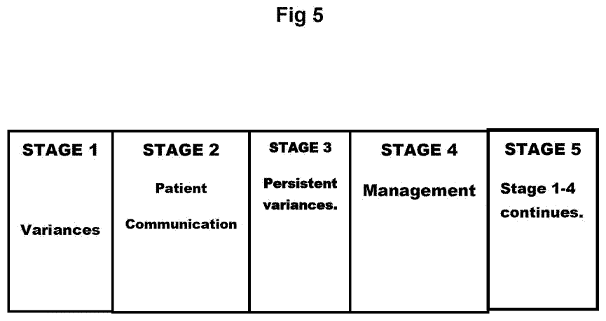

In summary, these are the variables sent either daily, quarterly, Bi annually or Annually to the Cloud data base for each patient through their UPID (Unique Patient ID), The daily data is transmitted through the Cell phone Application Ref FIG. 11.
Delivery of patient variance monitoring services is carried out through the 12 clinical parameters transmitted from the patients home, Labs or LTC to the IPCDB accessed and then displayed by the Comprehensive Digital Format on the G2H website enabling the MDT (Multi disciplinary Team) to effect the clinical transformation. Ref FIGS. 1-11.

We make the point that the process is designed to be patient centric and patients normally complete all steps on the Cell Phone App within 60 seconds after a days practice.

The formats below are the basis for health staff to make informed decisions after the data is transmitted from the Cell Phone App or from Labs to the IPCDB and displayed on the Comprehensive risk format hosted on the G2H website.

FIGS. 12 and 13

Daily Dashboard at FIG. 12 highlighting glucose levels (in red) above or within pre set levels with additional specific details of diet, Insulin Brand and Timing, Exercise and GIR %, Sample of the “Daily Dashboard” for large patient groups and the “Did not Test” Tab identifying patients on the call list. FIG. 13

The medical alert represents the first stage of the management process triggering a call from the Physicians office or hospital to the patient. Based on the call the Physician, Nurse or Diabetic Educator can review Insulin dosage, Diet and manage both Low and High Glucose levels.

Details of Diet, Exercise, Insulin Dosage and Timing, Test Time & Messaging after data is generated by the T1D Patient to the Cloud data base and displayed in one view of the comprehensive risk format hosted on the G2H website. With a direct relationship between Carbs and Glucose level displayed on 08/30. Ref FIGS. 8 and 9

The Multidisciplinary Team (MDT) consisting of Physicians, Diabetic Educators, Nurses and Mobile Support are provided with the management tools and format through access to the Integrated Patient Culinical Data Base (IPCDB) and the ability to remotely manage variances from clinical standards and thresholds. These features are the solution to delivering and managing large numbers of patients using an integrated model of care for T1D. Delivery is at the low cost Home, reducing high cost hospital admissions, re-admissions and use of the ER.

The patient centric design and the focus on ICR, ISF, and the format enables staff to get ahead of the disease.

The invention addresses each of the above points, meets the strategic initiatives of reducing costs in delivery of health care at low cost locations and ensures that the patient creates equity in the management of their own health.

FIGS. 14, 15 and 16:

The operations described above are compared at FIGS. 14, 15, 16 setting out the distinction between the G2H Type 1 Diabetes Monitoring Solution and Prior Art.

FIGS. 17, 18, 19, 20 and 21

These drawings and flow charts set out the process to commence patient monitoring, the Registration procedure and activating the Cell Phone App. Details are furnished at the next section: “Detailed description of the Invention”. To commence, it is necessary to complete the following steps which include documentation and registration in order for the Clinical Management Business process to be made available to the members of the MDT and Patient. We make the point that the process is designed to be patient centric and patients normally complete all steps on the Cell Phone App within 60 seconds after a days practice.

Ref FIGS. 17, 18, 19, 20, 21 set out the steps to commence monitoring.
1) Identification of patients: Clinical History, Cell Phone and Internet availability.
2) Explain Project details, Provide Information document, Complete “Consent Form”
3) Staff or Patient complete Registration, Generate UPID, Download App to patients Cell Phone, Patient Training on use of the Cell Phone Application and Web Site.
4) Do a Trial while the patient is with the Educator. Commence Testing and Monitoring Operations

FIGS. 22, 23, 24, 25, 26, 27, 28, 29

The description corresponding to these drawings is covered in the next section under “Detailed description of the Invention”. The content and drawing describe the process of the patient entering clinical data to the Cell phone app, transmitting it to the cloud and the formats displayed for Health Care Staff to manage patient data.

FIGS. 30, 31, 32, 33, 34

These Drawings are demonstrated examples of the output from External Sources and management relating to Prescriptions, Imaging, Pathology and Messaging. The method of transmission has been previously specified at FIG. 7. While the messaging feature is built in to the Solution permitting communication between the Patient and Health Care staff.

FIGS. 35, 36, 37 These drawings are a summary of clinical outcomes from Remote Monitoring projects for Type 1 Diabetics implemented overseas. The methodology was to provide the Cell Phone app to one set of patients (Group A) and compare the results with another Group B that did not have access to the App. There were very encouraging positive outcomes for the Group A patients compared to Group B.

FIGS. 38 and 39

These drawings set out the benefits to both patients and Payors in utilizing the Low Cost Solution in both the U.S. (Rural and Urban Markets) and Emerging economies.

Utilizing high speed internet, or cellular communication clinical data is transmitted from the home or labs to create a daily dashboard for the Physician to review variances from thresholds and access patient clinical history and other components of patient clinical information.

Physicians, Diabetic Educators, Nurses and the patients access the comprehensive digital format by accessing the IPCDB data in the Cloud.

### DETAILED DESCRIPTION OF THE INVENTION

To commence, it is necessary to complete the following steps which include documentation and registration in order for the Clinical Management Business process to be made available to the members of the MDT and Patient. We make the point that the process is designed to be patient centric and patients normally complete all steps on the Cell Phone App within 60 seconds after a days practice.

### Ref FIGS. 17, 18, 19, 20, 21

1) Identification of patients: Clinical History, Cell Phone and Internet availability.
2) Explain Project details, Provide Information document, Complete “Consent Form”
3) Staff or Patient complete Registration, Generate UPID, Download App to patients Cell Phone, Patient Training on use of the Cell Phone Application and Web Site.
4) Do a Trial while the patient is with the Educator. Commence Testing and Monitoring Operations

Patient Registration: This activity is carried out on the G2H website at the “Register Patient” Tab. Staff register patient details and create a Unique Patient ID (UPID) to be subsequently used for transmitting clinical data from all sources. The registration process—Please refer FIG. 18 is used for all sources of patient data including the network generated UPID, personal details, threshold values for medical alerts set by the physician and Care Plan. Other important parameters include the Project, Hospital, Brands of Insulin, Tablets, ICR, ISF and the Doctor responsible for the patient.

The registration is done by members of the MDT. with MPIN set by the patient, The user type can be the Patient in their home (Individual), Family (Multiple family members using one cell phone) or Group (In a Long Term Care or Assisted Living facility)
The UPID is also used by the patient to sign in on their Cell Phone together with the MPIN to acquire and transmit data to the IPCDB.
The Insulin Brands (in this case two brands) are selected from the registration menu together with (in this case) one tablet from the corresponding tablet menu. This selection is subsequently automatically replicated on the Cell Phone application. Insulin Brands are selected from the Menu based on the Doctors Prescription during Registration. Tablet Brand selected from the Menu during Registration based on the Doctors Prescription. Ref FIG. 19
The registration process generates a Patient ID (UPID) used together with the MPIN created by the patient to start operation of the Cell Phone Application.

The Cell phone application is downloaded from the Google Play Store (Gateway2Health g2h.care) and installed on the patients Cell Phone. After opening the application on the cell phone, the UPID and MPIN are entered for the first time and remain in cell phone memory for subsequent tests. In this case the Patient ID is 00179 and MPIN set by the patient is entered Ref FIG. 20

After entering the UPID and MPIN the operating app appears on the Patient Cell Phone together with the patient name. FIG. 21

Once the Registration process is complete there are two possible scenarios
1) The Patient sending current glucose test results following prior Insulin Shots, Exercise and a meal or
2) Anticipation of spikes in Glucose on special occasions or alternately advice from the Diabetes Educator to correct glucose levels through increases (or deceases) in Insulin dosage or Insulin brands through Insulin shots in advance of the meal or special occasion.
   Both scenarios are set out below:

Patient sends current glucose test results based on prior Insulin shots, Meals and Exercise. Please Ref FIG. 22

The date of the Glucose Test is extracted from the Cell Phone Settings and changes automatically. The patient then clicks on each of the tabs commencing in the order that the activity occurred starting from the time of glucose test.—in this case 2 hours after breakfast at 9 AM. followed by Exercise at the Gym for 30 Minutes before breakfast

The details of the previous meal (breakfast) are selected from the “Latest Meal” Menu with Carbs automatically Calculated. (48 Carbs). FIGS. 23 and 24

Selection: Cooked Oats, 2 Scrambled Eggs, Coffee with Milk, Orange Juice: 48 Carbs and 16 g proteins calculated automatically. FIG. 23

The Insulin Brands selected during Registration are linked to the patients ID and appear on the Cell Phone. Novomix 30 is selected for the Insulin injection before breakfast. The actual time of the insulin injection is then checked at 6.30 AM FIG. 25

The final screen on the Cell Phone with the Insulin, Tablets, Test Time, Diet and Exercise is displayed on the screen prior to “Save” and “Close” which transmit the data linked to the UPID to the Cloud Data Base after the glucose test at 9 AM. Ref FIG. 26 Final Screen—Insulin Before Breakfast. Test Time two Hours after breakfast.

A similar sample exercise is displayed below for a special occasion or changes in insulin where the patient knows that the Carb intake is likely to be greater the target. We note that the baseline Insulin dose for Dinner is 6 Units. And the glucose test before dinner is 152 mg/dl which is already above target.

The “ Add Insulin ” option has to be selected. Please refer FIG. 28
In this case the Base Insulin Units at Dinner are 6 Units and the ICR/ISF calculations require an additional 5 Units to control the spike due to the 110 Carbs estimated at the special occasion. (Target Carbs 75).
The Patient therefore injects 11 Units (Baseline 6 Plus additional 5 Units) Before the special occasion to offset the impact of the Carb intake of 110 against the target of 75.

Access to the data in the Cloud by the Physician, Diabetic Educator or the patient is through the comprehensive format hosted on the G2H web site providing all the above details transmitted by the patient from home via cellphone to the cloud or uploaded through the “Clinical Data Tab” Both data categories form part of Medical records. Ref FIGS. 27 and 29

We have described above:
1. The Glucose level in the morning at 9 AM of 128 mg/dl 2 hours after a breakfast of 48 Carbs which is retrospective relating to Insulin Shots, Exercise and Breakfast at 7 AM.
2. Additional Insulin required for a special occasion dinner estimated at 110 Carbs and therefore requiring 11 Units before the meal to offset the impact of the increased carb levels.

Clicking the other Tabs at the top of the Screen provides access to Prescriptions, Imaging Docs, Pathology, Graphs and Messaging. FIGS. 30, 31, 32, 33, 34

To summarize, the invention comprises, a specially designed Cell Phone App, linked to a business process which integrates Data on a single platform and displays multiple formats enabling a Clinical transformation consequent to the clinical Management of T1D not achievable by the Human Mind or Computer acting independently.

Results of the Remote Monitoring Program directed to T1D patients at an overseas hospital. Please refer to FIGS. 35, 36, 37

The Business Process uses Technology to transmit Glucose readings from the patients home through an application on their cell phone, calculates variances from pre set thresholds (70-180 mg/dl) based on 3-4 tests every day with the Diabetes Educator and Physician being able to monitor both the readings and variances remotely at their Hospital. The Experimental Group (Group A) was made up of 17, Type 1 Diabetic Patients provided with the Cell Phone Application to transmit Glucose levels to a Cloud Data Base while the corresponding Control Group of 15 patients (Group B) was required to transmit Glucose levels through a traditional process through an excel spreadsheet or phone calls at their regular frequencies of once every 3-4 days. Both groups were required to test 3 times a day.
The project appointed a dedicated Diabetic Educator to act as a clinical monitoring and clinical management support resource available to patients or their parents for both Experimental and Control Groups.
Patients in both Groups A & B lived between 7 KM (4 Miles)—224 KM (140 Miles) from the Hospital.
The discipline of a testing regime was reflected in 4590 Tests done by Group A and 4797 tests by Group B to provide a total of 9387 Data Points for Analysis.
The summary of the project results demonstrated improved performance in glucose control for the Experimental Group A provided with the Cell Phone Application.
a) The appointment of a dedicated Diabetic educator was welcomed by both Groups representing a single resource who could be contacted by patients or their parents.
b) The Daily dashboard was a very useful risk format used by the educator to review results from the previous night and during the course of the day and especially focused on Hypos and Insulin Dosage.
c) During the first month, the communication traffic was mainly from the educator to the patients (Group A). A positive feature emerged during the second and third month where 5-6% of the communications were from patients to the educators, requesting support on managing glucose.
d) Communication for Group B was primarily from Educator to Patient and on a few occasions the educator had to request the patients to send their results.
e) Feedback from Group A was positive in terms of improved communication, managing their medical condition and improving quality of life.

OUTCOME SUMMARY. Please refer to FIGS. 35, 36. 37.
Mean Glucose: The average of glucose readings for the Group over 100 Days with glucose tested 3 times a day.
Coefficient of Variation (CV %) is the “Standard Deviation” of the Mean Glucose for each patient divided by the Mean to reduce Volatility of mean Glucose Levels for each patient. Physicians set a target of 33%
Physicians use GIR % (Glucose in Range %) which is the Cumulative Percentage of readings within the Target 70-180 mg/dl.
Physicians also use a tighter glucose band of 70-154 mg/dl to monitor and manage patients. The difference in glucose review time cycles by the dedicated monitoring center of Group A (Daily) compared with Group B (every 3-4 days) translated to better control with mean levels in the tighter 70-154 mg/dl range—reflected in 10 patients out of 17 in Group A (58%) versus 5 out of 15 (33%) in Group B.

Low cost Solution directed to vulnerable population segments. Please Ref FIGS. 38 and 39

The Drawings set out a comprehensive cost of service delivery including all major cost components—the calculations take into account that the patient has a glucometer and Insulin. Likely sponsors are the Medicare and Medicaid programs, Diabetes Networks, International Aid agencies or Hospitals as a mechanism of deriving On Line Revenue. The commercial basis is the savings on hospitalization or ER visits compared to the annual cost of the programs. Hospitalization costs for Type 1 Diabetic patients are relatively higher because of the complex nature of the disease with indicative costs between $6000-$9,000 a Day.

A similar program delivered in a developing country like India which has the highest number of Diabetic Patients worldwide is significantly less expensive because of lower Physician, Nurses and Diabetic Educator Costs. (Exchange rate 75 Indian rupees=1 USD)

Invention Executive Summary: Gateway 2Health (G2H) Remote Monitoring for Managing Type 1 Diabetes (T1D)

There is a critical and urgent need to direct resources to the management of this clinical sub segment.

The invention comprises, a specially designed Cell Phone App, linked to a business process which integrates 12 parameters of patient clinical data on a single digital platform and displays multiple digital formats for viewing by health care staff and the patient.
The combination of technology and clinical features does more than aggregate the elements of prior art and leads to clinical outcomes and transformations that are more than and provide enhanced positive clinical results when compared to a predictable result from mere combination of elements.
Clinical Outcomes for patient groups using the invention demonstrate enhanced and positive transformation compared to groups without the invention.
The invention is not directed to any abstract ideas, laws of nature and natural phenomena (including products of nature). The process required in the invention does not require and is not directed to any abstract ideas since the mental process in the mind of health care staff in the review and final step is primarily based on clinical evidence produced independently and separately through the business process, it is the combination rather than the independent operation of the Mind or the display of data using the Computer technology that leads to clinical transformation.
This invention to monitor Type 1 Diabetics represents two distinct stages comprising a) the transmission of clinical data through various standard technologies to the cloud and b) subsequent access of the data using a browser to display some or all data, variances or changes in pre set formats and review and management of the data by Health Care Staff.
The invention therefore has two distinct components with the “Person having ordinary skill in the art” being actually two different persons with two different skill sets.—Technology to transmit clinical data, and Health Care clinical know how to review the data and does not meet the “Obvious” criterion.

1-2. (canceled)

3. The Method using standard technology protocols to link patient and project ID with glucose management metrics transmitted to a cloud data base using a unique Digital Application (App) on each patients cell phone and including Web Site data entry enabling the subsequent simultaneous display of patient data comprising 12 clinical Parameters in specially designed formats applied to managing Type 1 Diabetes (T1D).
The method comprising the display of the said 12 clinical parameters in specially designed formats;
through in the first instance generating at Registration a Unique Patient ID (UPID) to link patient data including glucose readings, medication, diet, exercise, pathology, imaging and Prescription details;
using standard technology protocols to transfer glucose readings and glucose metrics, diet, exercise and insulin brand and units details multiple times a day to a cloud data base from the Cell Phone App
displaying through Web site based entry, details of pathology tests, retina scan imaging and prescriptions at frequencies dependent on Clinical Care schedules;
display the clinical data in predesigned clinical management formats including diet including carbs and protein, insulin brand and units, exercise details and intensity and, after calculation, Mean Glucose, glucose variances, Glucose in Range (GIR %), and Coefficient of Variation (CV %), carbs and proteins representing one part of the invention;
the predesigned formats enable the Health Care professional, Doctor, Nurse or Educator to perform a series of interventions or series of acts;
these interventions can occur multiple times a day to control glucose levels and glucose volatility through the specially designed formats and effect clinical transformation and is the Second part of the invention;
enabling health professionals to simultaneously and concurrently manage T1D patient groups or individuals;
carry out multiple clinical interventions (Acts or a series of acts, performed upon the subjects to be transformed) for patient groups or individuals within these groups with Type 1 Diabetes (T1D);
within a single digital platform;
to effect a clinical transformation and deliver enhanced clinical results compared to a predictable result from mere combination of elements for patients who are not provided with the invention;
requiring two separate and distinct skills comprising an individual skilled in the art of the method to display results in predesigned formats and a second individual skilled in the art of Health Care to interpret the displays, multiple times as part of the clinical transformation.
The said 12 Parameters include:
1) Time of Glucose Test, and Glucose levels
2) Insulin Brand
3) Insulin Dosage and Time
4) Brand of Tablets
5) Exercise details and intensity
6) Carbohydrates and Proteins
7) Glucose in Range (GIR %)
8) ICR, (Insulin Carbohydrate Ratio)
9) ISF (Insulin Sensitivity Factor)
10) Pathology
11) Prescriptions
12) Imaging of Diabetic Retina Scans.

4. The Business method in claim 1 to provide health care staff with clinical patient information on how the 12 parameters relate to each other at different points in time;
the predesigned format display includes current and historical glucose levels by date and time, time and quantum of insulin, dosage, Diet, carbs and protein intakes, Exercise levels and intensity.
and after calculations, mean glucose, variances from pre set thresholds, Coefficient of variation (CV %), Glucose in Range GIR %, Carbs and Proteins;
separate Tabs on the single digital platform provide health Care staff with access to Pathology reports, Prescription details and Retina scans with
enabling health Care Staff to review all parameters multiple times a day to take appropriate intervention action
multiple Interventions leads to clinical transformations and enhanced clinical results compared to a predictable result from mere combination of elements for patients who are not provided with the invention
